SEQUENCE LISTING (32 sequences)

SEQ ID NO: 1 - DNA, 660 bp, Arabidopsis thaliana
agtactatactacaacatttccatattttttttagattgttagctaatttcccctggagataattgtaaattgtttcaatgagaggaatatacaatacatagatcgtaattgatcaatgcgtatttgcatgttaatacatttgtgtcttgtaccaaaaaaaggaattatacatttgtgtcatttaactctggacaccatacatttcgtcattacagtgaaacggcagaatttgaacactcattattctggttggtagttatttccatatttctcaaagaacatttatgtgactattatcattccttgcgacaactgtaataatgagaaaacttggtattttttttggctcttcatataagttgtttaaaaaatgtattttcaaacaataattaatagtggcccaaacataatttaaaataggtttcgaagcccaaagcccataaattaaacgcctaacattcacgcgctctttgactatggttgcttaggaacagatgcgcgtggggaagttggcaccgttttttctcttgcttacatctatttttttttcttaaacgtctatttatttgctttacgtcattgtaacgtttgtttgttttctctgtatcgttagttgttgtacacttgtactatggacgttgaactgcggttagtccggt

SEQ ID NO: 2 - DNA, 662 bp, Arabidopsis thaliana
agtactatactacaacatttccatattttttttagattgttagctaatttcccctggagataattgtaaattgtttcaatgagaggaatatacaatacatagatcgtaattgatcaatgcgtatttgcatgttaatacatttgtgtcttgtaccaaaaaaaggaattatacatttgtgtcatttaactctggacaccatacatttcgtcattacagtgaaacggcagaatttgaacactcattattctggttggtagttatttccatatttctcaaagaacatttatgtgactattatcattccttgcgacaactgtaataatgagaaaacttggtattttttttggctcttcatataagttgtttaaaaaatgtattttcaaacaataattaatagtggcccaaacataatttaaaataggtttcgaagcccaaagcccataaattaaacgcctaacattcacgcgctctttgactatggttgcttaggaacagatgcgcgtggggaagttggcaccgttttttctcttgcttacatctatttttttttcttaaacgtctatttatttgctttacgccattgtaacgtttgtttgttttctctgtatcgttagttgttgtacacttgtactatggacgttgggaactgcggttagtccggt

SEQ ID NO: 3 - DNA, 662 bp, Arabidopsis thaliana
agtactatactacaacatttccatattttttttagattgttagctaatttcccctggagataattgtaaattgtttcaatgagaggaatatacaatacatagatcgtaattgatcaatgcgtatttgcatgttaatacatttgtgtcttgtaccaaaaaaaggaattatacatttgtgtcatttaactctggacaccatacatttcgtcattacagtgaaacggcagaatttgaacactcattattctggttggtagttatttccatatttctcaaagaacatttatgtgactattatcattccttgcgacaactgtaataatgagaaaacttggtattttttttggctcttcatataagttgtttaaaaaatgtattttcaaacaataattaatagtggcccaaacataatttaaaataggtttcgaagcccaaagcccataaattaaacgcctaacattcacgcgctctttgactatggttgcttaggagcagatgcgcgtggggaagttggcaccgttttttctcttgcttacatctatttttttttcttaaacgtctatttatttgctttacgtcattgtaacgtttgtttgttttctctgtatcgttagttgttgtacacttgtactatggacgttgaaaactgcggttagtccggt

SEQ ID NO: 4 - DNA, 662 bp, Arabidopsis thaliana
agtactatactacaacatttccatattttttttagattgttagctaatttcccctggagataattgtaaattgtttcaatgagaggaatatacaatacatagatcgtaattgatcaatgcgtatttgcatgttaatacatttgtgtcttgtaccaaaaaaaggaattatacatttgtgtcatttaactctggacaccatacatttcgtcattacagtgaaacggcagaatttgaacactcattattctggttggtagttatttccatatttctcaaagaacatttatgtgactattatcattccttgcgacaactgtaataatgagaaaacttggtattttttttggctcttcatataagttgtttaaaaaatgtattttcaaacaataattaatagtggcccaaacataatttaaaataggtttcgaagcccaaagcccataaattaaacgcctaacattcacgcgctctttgactatggttgcttaggaacagatgcgcgtggggaagttggcaccgttttttctcttgcttacatctatttttttttcttaaacgtctatttatttgctttacgtcattgtaacgtttgtttgttttctctgtatcgttagttgttgtacacttgtactatggacgttgaaacctgcggttagtccggt

SEQ ID NO: 5 - DNA, 43 bp, Artificial Sequence
  Description of Artificial Sequence  Synthetic Primer
taggagactgacatagattggaggagacattttgtataatagg

SEQ ID NO: 6 - DNA, 80 bp, Artificial Sequence
  Description of Artificial Sequence  Synthetic Primer
aggagactgacatagattggaggagacattttgtataataaggagactgacatagattggaggagacattttgtataata

SEQ ID NO: 7 - DNA, 129 bp, Artificial Sequence
  Description of Artificial Sequence  Synthetic Primer
aggagactgacatagattggaggagacattttgtataataaggagactgacatagattggaggagacattttgtataataactagctaggagactgacatagattggaggagacattttgtataatagg

SEQ ID NO: 8 - DNA, 166 bp, Artificial Sequence
  Description of Artificial Sequence  Synthetic Primer
aggagactgacatagattggaggagacattttgtataataaggagactgacatagattggaggagacattttgtataataactagcaggagactgacatagattggaggagacattttgtataataaggagactgacatagattggaggagacattttgtataata

SEQ ID NO: 9 - DNA, 215 bp, Artificial Sequence
  Description of Artificial Sequence  Synthetic Primer
aggagactgacatagattggaggagacattttgtataataaggagactgacatagattggaggagacattttgtataataactagcaggagactgacatagattggaggagacattttgtataataaggagactgacatagattggaggagacattttgtataataactagctaggagactgacatagattggaggagacattttgtataatagg

SEQ ID NO: 10 - DNA, 252 bp, Artificial Sequence
  Description of Artificial Sequence  Synthetic Primer
aggagactgacatagattggaggagacattttgtataataaggagactgacatagattggaggagacattttgtataataactagcaggagactgacatagattggaggagacattttgtataataaggagactgacatagattggaggagacattttgtataataactagcaggagactgacatagattggaggagacattttgtataataaggagactgacatagattggaggagacattttgtataata

SEQ ID NO: 11 - DNA, 20 bp, Artificial Sequence
  Description of Artificial Sequence  Synthetic Primer
accagcctttgtttcaacaa

SEQ ID NO: 12 - DNA, 26 bp, Artificial Sequence
  Description of Artificial Sequence  Synthetic Primer
cataatcaataagaaaatagatgtac

SEQ ID NO: 13 - DNA, 45 bp, Artificial Sequence
  Description of Artificial Sequence  Synthetic Primer
aaagaattcaactagtaggatccagtactatactacaacatttcc

SEQ ID NO: 14 - DNA, 36 bp, Artificial Sequence
  Description of Artificial Sequence  Synthetic Primer
aaagcggccgcccgggaccggactaaccgcagttca

SEQ ID NO: 15 - DNA, 70 bp, Artificial Sequence
  Description of Artificial Sequence  Synthetic Primer
aaagaattcgctagcaggagactgacatagattggaggagacattttgtataataaggagactgacatag

SEQ ID NO: 16 - DNA, 70 bp, Artificial Sequence
  Description of Artificial Sequence  Synthetic Primer
aaaggatccgatggccgcactagttattatacaaaatgtctcctccaatctatgtcagtctccttattat

SEQ ID NO: 17 - DNA, 49 bp, Artificial Sequence
  Description of Artificial Sequence  Synthetic Primer
ctagctaggagactgacatagattggaggagacattttgtataatagga

SEQ ID NO: 18 - DNA, 49 bp, Artificial Sequence
  Description of Artificial Sequence  Synthetic Primer
ctagtcctattatacaaaatgtctcctccaatctatgtcagtctcctag

SEQ ID NO: 19 - DNA, 51 bp, Artificial Sequence
  Description of Artificial Sequence  Synthetic Primer
ctagcatttatttatttatttatttatttatttatttatttatttatttag

SEQ ID NO: 20 - DNA, 51 bp, Artificial Sequence
  Description of Artificial Sequence  Synthetic Primer
gatcctaaataaataaataaataaataaataaataaataaataaataaatg

SEQ ID NO: 21 - DNA, 61 bp, Artificial Sequence
  Description of Artificial Sequence  Synthetic Primer
ctagcatttaatttaatttaatttaatttaatttaatttaatttaatttaatttaatttag

SEQ ID NO: 22 - DNA, 61 bp, Artificial Sequence
  Description of Artificial Sequence  Synthetic Primer
gatcctaaattaaattaaattaaattaaattaaattaaattaaattaaattaaattaaatg

SEQ ID NO: 23 - DNA, 51 bp, Artificial Sequence
  Description of Artificial Sequence  Synthetic Primer
ctagcatgaatacatctgaatgtctagtatattgattgaaagctttgtatg

SEQ ID NO: 24 - DNA, 51 bp, Artificial Sequence
  Description of Artificial Sequence  Synthetic Primer
gatccatacaaagctttcaatcaatatactagacattcagatgtattcatg

SEQ ID NO: 25 - DNA, 61 bp, Artificial Sequence
  Description of Artificial Sequence  Synthetic Primer
ctagcatctgatactgacatgcatcatgctaattcagacatgcatgaattcaatacgtacg

SEQ ID NO: 26 - DNA, 61 bp, Artificial Sequence
  Description of Artificial Sequence  Synthetic Primer
gatccgtacgtattgaattcatgcatgtctgaattagcatgatgcatgtcagtatcagatg

SEQ ID NO: 27 - DNA, 13 bp, Artificial Sequence
  Description of Artificial Sequence  Synthetic Primer
atttatttattta

SEQ ID NO: 28 - DNA, 21 bp, Artificial Sequence
  Description of Artificial Sequence  Synthetic Primer
atttatttatttatttattta

SEQ ID NO: 29 - DNA, 56 bp, Artificial Sequence
  Description of Artificial Sequence  Synthetic Primer
atttaatttaatttaatttaatttaatttaatttaatttaatttaatttaatttaa

SEQ ID NO: 30 - DNA, 33 bp, Artificial Sequence
  Description of Artificial Sequence  Synthetic Primer
atttatttatttaatttaatttaatttatttaa

SEQ ID NO: 31 - DNA, 16 bp, Artificial Sequence
  Description of Artificial Sequence  Synthetic Primer
atttaatttaatttaa

SEQ ID NO: 32 - DNA, 26 bp, Artificial Sequence
  Description of Artificial Sequence  Synthetic Primer
atttaatttaatttaatttaatttaa

(19) United States
(12) Patent Application Publication
(10) Pub. No.: US 2007/0011761 A1
(43) Pub. Date: Jan. 11, 2007

(54) Post-transcriptional regulation of gene expression

(75) Inventors: Kwan Y. Thai, O'Fallon, MO (US); Qi Wang, St. Louis, MO (US); John P. Dabrowski, Rock Hill, MO (US); Tim N. Ulmasov, Chesterfield, MO (US); Ida M. House, Edwardsville, FL (US)

Correspondence Address: FULBRIGHT & JAWORSKI, LLP, 600 CONGRESS AVENUE, SUITE 2400, AUSTIN, TX, 78745, US

(73) Assignee: MONSANTO TECHNOLOGY, L.L.C.

(21) Appl. No.: 11/434,556

(22) Filed: May 15, 2006

Related U.S. Application Data
(60) Provisional application No. 60/682,471, filed on May 19, 2005.

(51) Int. Cl. A01H 5/00; C12N 15/82; C12N 5/04; A01H 1/00

(52) U.S. Cl. 800/278; 800/312; 800/320.1; 435/468; 435/412; 435/415

(57) ABSTRACT

The invention provides transgenic plants having within their genome DNA that includes an exogenous gene encoding a polypeptide and having in its 3′ untranslated region a destabilizing sequence, whereby the polypeptide is expressed at a lower level in seed of the transgenic plants relative to expression in the absence of the destabilizing sequence. Also disclosed are methods for post-transcriptionally decreasing the message stability of a gene of interest in a plant that involve adding a destabilizing sequence to the 3′ untranslated region of the gene of interest.

This application claims the benefit of the filing date of U.S. provisional patent application Ser. No. 60/682,471, filed May 19, 2005, the entire disclosure of which is specifically incorporated herein by reference.

### BACKGROUND OF THE INVENTION

1. Field of the Invention

The present invention relates generally to plant molecular biology, and more specifically to methods for post-transcriptional regulation of gene expression in plants.

2. Background of the Invention

In the field of biotechnology, a major focus has been on increasing the expression level of transgenes, for example, to achieve a specific commercial target. Molecular elements that down-regulate gene expression have been largely overlooked because they have been generally perceived as having no commercial value. Nonetheless, elements that down-regulate gene expression, especially elements that down-regulate gene expression in a predictable manner, can be useful, particularly when it is desirable to achieve a specific level of gene expression. For example, very high expression of a given gene is not always desirable, and one may wish to achieve expression of a given transgene above one level yet below another level. While gene expression can be regulated at the transcription step (for example, by the selection of appropriate promoters or promoter elements to be used with a given transgene), it is also possible to post-transcriptionally regulate gene expression. Such post-transcriptional control of gene expression also offers the advantage of still allowing one to use the temporal, spatial, or inducibility profiles obtainable by use of appropriate promoters or promoter elements.

One approach to post-transcriptional control of gene expression is to control the stability of the messenger RNA (mRNA) produced by gene transcription. Including a destabilizing sequence in the 3′ untranslated region (3′ UTR) of a transcribed RNA has been shown to destabilize mRNA and reduce mRNA half-life in tobacco (Nicotiana tabacum) and in animal RNAs. For example, the SAUR (small auxin up RNAs) genes of plants contain the DST element, a 43 base pair sequence in the 3′ untranslated region (3′ UTR) highly conserved across plant species, which has been reported to confer instability to SAUR mRNAs at least in tobacco plants. See, e.g., Newman et al. (1993), Green (1993), and Gutiérrez et al. (1999), and Feldbrugge et al. (2001), which are incorporated by reference herein. It was not known if the SAUR terminator or the DST element would have similar effects on RNAs from dicot crops or monocot plants.

Another conserved RNA motif, multiple copies of AUUUA, is believed to destabilize mRNAs in animals and is also found in plants; AUUUA repeats were reported to destabilize mRNAs in tobacco whereas AUUAA repeats did not, indicating sequence specificity for this motif and not just AU content. See, for example, Ohme-Takagi et al. (1993), and Gutiérrez et al. (1999), which are incorporated by reference herein. However, it was not known if the AUUUA repeat would have similar effects in other dicots or in monocot plants.

### SUMMARY OF THE INVENTION

The present invention provides a method of post-transcriptionally regulating gene expression in a plant, such as dicot crop plants and monocot crop plants. More specifically, the present invention discloses a method of post-transcriptionally decreasing message stability in a plant, including adding a destabilizing sequence to the 3′ untranslated region of a gene of interest in the plant, whereby message stability of the gene of interest is post-transcriptionally decreased, preferably resulting in expression of the gene of interest at a lower level relative to that seen where the destabilizing sequence is not present.

In one aspect, the present invention provides a transgenic plant having in its genome DNA comprising an exogenous gene encoding a polypeptide and having in its 3′ untranslated region one or more destabilizing sequences, whereby the polypeptide is expressed at a lower level in seed of the transgenic plant relative to expression in the absence of the one or more destabilizing sequences.

In another aspect, the present invention claims a transgenic plant having in its genome DNA including a non-constitutive promoter operably linked to an exogenous gene encoding a polypeptide and having in its 3′ untranslated region one or more destabilizing sequences, whereby the polypeptide is expressed at a lower level in the transgenic plant relative to expression in the absence of the one or more destabilizing sequences.

In a further aspect, the present invention claims a transgenic crop plant used for food or feed and having in its genome DNA including an exogenous gene encoding a polypeptide and having in its 3′ untranslated region one or more destabilizing sequences, whereby the polypeptide is expressed at a lower level in the transgenic crop plant used for food or feed relative to expression in the absence of the one or more destabilizing sequences.

In yet another aspect, the present invention claims a transgenic plant having in its genome DNA comprising an exogenous gene encoding a polypeptide and having in its 3′ untranslated region one or more destabilizing sequences including overlapping ATTTAA repeats, whereby said polypeptide is expressed at a lower level in said transgenic plant relative to expression in the absence of said one or more destabilizing sequences.

In yet another aspect, the present invention further claims a transgenic plant having in its genome DNA including a gene encoding anthranilate synthase and having in its 3′ untranslated region one or more destabilizing sequences, whereby the anthranilate synthase is expressed at a lower level in the transgenic plant relative to expression in the absence of the one or more destabilizing sequences.

The present invention also provides methods to post-transcriptionally decrease message stability of a gene of interest in a crop plant used for food or feed. In one embodiment, the method includes adding one or more destabilizing sequences to the 3′ untranslated region of the gene of interest in the crop plant used for food or feed, whereby message stability of the gene of interest is post-transcriptionally decreased and preferably results in expression of the gene at a level lower than that where the one or more destabilizing sequence is absent.

Other specific embodiments of the invention are disclosed in the following detailed description.

### BRIEF DESCRIPTION OF THE DRAWINGS

FIG. 1. Non-limiting examples of constructs containing destabilizing sequences as used in the transient transformation experiments described in the examples below. An overall design is illustrated and pertinent elements of the constructs are listed.

FIG. 2. A non-limiting example of destabilizing elements useful in lowering the level of expression of a gene or polypeptide as described in Example 2. Different variants of SAUR terminators (pMON63688) effectively decreased gene expression as compared with NOS terminator (pMON63691) in constructs using Lea9 promoter driving GUS gene.

FIG. 3. A non-limiting example of destabilizing elements useful in lowering the level of expression of a gene or polypeptide as described in Example 2. The SAUR terminator (pMON63688, variant 1) effectively decreased gene expression as compared with NOS terminator (pMON13773) in constructs using 7Salpha′ promoter driving GUS gene.

FIG. 4. A non-limiting example of destabilizing elements useful in lowering the level of expression of a gene or polypeptide as described in Example 4. The 2× DST element in combination with NOS terminator (pMON63687) can effectively decrease gene expression as compared with NOS terminator alone (pMON58101) in constructs using USP promoter driving GUS gene.

FIG. 5. A non-limiting example of destabilizing elements useful in lowering the level of expression of a gene or polypeptide as described in Example 4. The 2× DST element in combination with NOS terminator (pMON63698) effectively decreased gene expression as compared with NOS terminator alone (pMON13773) in constructs using 7Salpha′ promoter driving GUS gene.

FIG. 6. A non-limiting example of destabilizing elements useful in lowering the level of expression of a gene or polypeptide as described in Example 4. The 2× DST element in combination with NOS terminator (pMON63697) effectively decreased gene expression as compared with NOS terminator alone (pMON63691) in constructs using Lea9 promoter driving GUS gene.

FIG. 7. A non-limiting example of destabilizing elements useful in lowering the level of expression of a gene or polypeptide as described in Example 8. Different copy numbers of DST element in combination with NOS terminators (pMON78113, pMON78116, pMON78117) effectively decreased gene expression as compared with NOS terminator alone pMON64316) in constructs using Per1 promoter driving GUS gene. The copy number of DST was negatively correlated with the level of gene expression.

FIG. 8. Non-limiting examples of constructs containing destabilizing sequences and useful for generating transgenic plants of the invention. An overall design is illustrated and key elements of the constructs are listed as described in Example 9.

FIG. 9 depicts non-limiting examples of destabilizing elements useful in lowering the level of expression of a gene or polypeptide as described in Example 9. DST and AU-rich elements effectively lowered gene expression in a transient maize expression system. The SAUR terminator contained 1× DST. Standard deviation is shown. The expression levels shown are relative to the control vectors which contained NOS terminator, spacer 1, or spacer 2.

FIG. 10 depicts non-limiting examples of destabilizing elements useful in lowering the level of expression of a gene or polypeptide as described in Example 10. Lower levels of tryptophan (Trp) were achieved in transgenic soybean by including destabilizing elements (DST motifs) to the 3′ untranslated region. The statistical software JUMP was used to generate the graph, which depicts results from individual R1 seed values of multiple events. The horizontal lines are the mean values of the Trp level which are given in parts per million (ppm). The circles shown in the column labeled “Each Pair Students t” represent the variability of the Trp level in each seed; the size of the circle indicates the degree of variability. Where none of the circles overlap another, the mean values are statistically significant difference from each other.

FIG. 11. Non-limiting examples of destabilizing elements useful in lowering the level of expression of a gene or polypeptide as described in Example 9. The lower levels of tryptophan (Trp) were correlated to less steady-state RNA levels in transgenic soybean, which demonstrates that including 2 copies of destabilizing elements (DST motifs) to the 3′ untranslated region is sufficient to decrease the transcripts. Immature seeds from two events of pMON63680 (−DST) and two events of pMON66892 (+DST) were harvested and total RNA was extracted using the conventional method. A portion of the RNA was used for quantifying the transcript level by Taqman and the other portion was used for northern analysis. Panel A shows the Transcript level by Taqman. For −DST, s1-s4 were plants from one event and s5-s7 were plants from a second event. For +DST, s8-s9 were plants from one event and s10-s15 were plants from a second event. Panel B shows relative transcript level on a northern blot. The Agro AS was used as a probe. The Taqman and the northern results are consistent with each other. The bottom of panel B shows the relative loading amount of RNA on the blot.

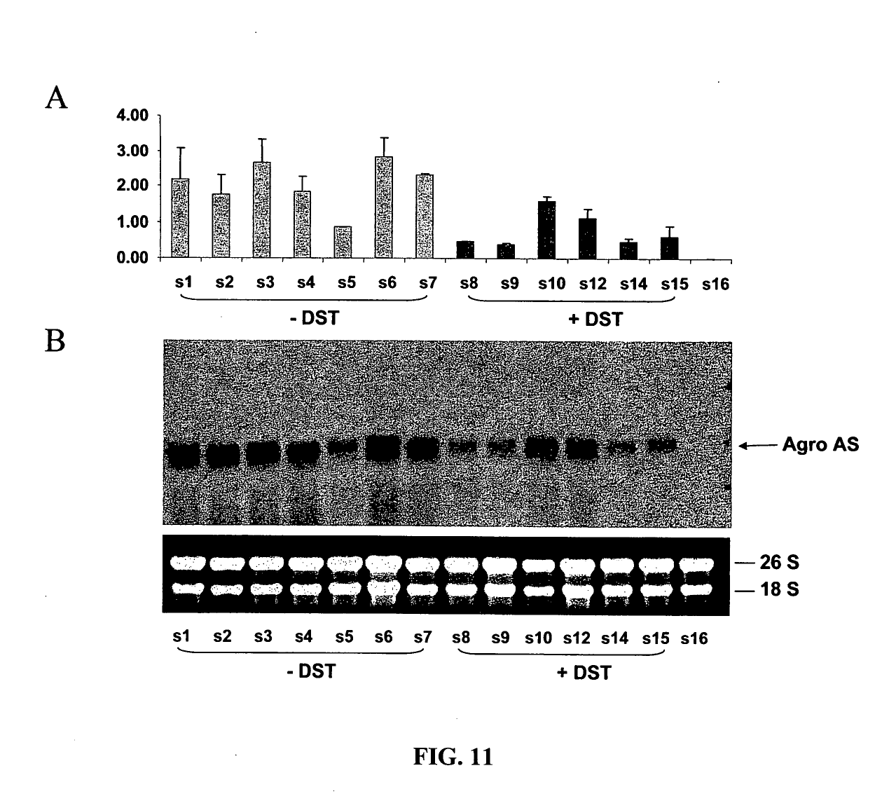

### DETAILED DESCRIPTION OF THE INVENTION

### I. TRANSGENIC PLANTS

The present invention provides a transgenic plant having in its genome DNA comprising an exogenous gene encoding a polypeptide and having in its 3′ untranslated region a destabilizing sequence, whereby the polypeptide is expressed at a lower level in seed of the transgenic plant relative to expression in the absence of the destabilizing sequence.

The transgenic plant may be derived from any monocot or dicot plant of interest, including, but not limited to, plants of commercial or agricultural interest, such as crop plants (especially crop plants used for human food or animal feed), wood- or pulp-producing trees, vegetable plants, fruit plants, and ornamental plants. Non-limiting examples of plants of interest include grain crop plants such as wheat, oat, barley, maize, rye, triticale, rice, millet, sorghum, quinoa, amaranth, and buckwheat; forage crop plants such as forage grasses and forage alfalfa; oilseed crop plants such as cotton, safflower, sunflower, soybean, canola, rapeseed, flax, peanuts, and oil palm; tree nuts (such as walnut, cashew, hazelnut, pecan, almond, and the like); sugarcane, coconut, date palm, olive, sugarbeet, tea, and coffee; wood- or pulp-producing trees; vegetable crop plants such as legumes (for example, beans, peas, lentils, alfalfa, peanut), lettuce, asparagus, artichoke, celery, carrot, radish, the brassicas (for example, cabbages, kales, mustards, and other leafy brassicas, broccoli, cauliflower, Brussels sprouts, turnip, kohlrabi), edible cucurbits (for example, cucumbers, melons, summer squashes, winter squashes), edible alliums (for example, onions, garlic, leeks, shallots, chives), edible members of the Solanaceae (for example, tomatoes, eggplants, potatoes, peppers, groundcherries), and edible members of the Chenopodiaceae (for example, beet, chard, spinach, quinoa, amaranth); fruit crop plants such as apple, pear, citrus fruits (for example, orange, lime, lemon, grapefruit, and others), stone fruits (for example, apricot, peach, plum, nectarine), banana, pineapple, grape, kiwifruit, papaya, avocado, and berries; and ornamental plants including ornamental flowering plants, ornamental trees and shrubs, ornamental groundcovers, and ornamental grasses. Preferred dicot plants include, but are not limited to, canola, cotton, potato, quinoa, amaranth, buckwheat, safflower, soybean, sugarbeet, and sunflower, more preferably soybean, canola, and cotton. In a particularly preferred embodiment, the transgenic plant is a transgenic monocot plant, more preferably a transgenic monocot crop plant, such as, but not limited to, wheat, oat, barley, maize, rye, triticale, rice, ornamental and forage grasses, sorghum, millet, and sugarcane, more preferably maize, wheat, and rice.

By “exogenous gene” is meant any gene that occurs out of the context in which it normally occurs in nature. Thus, an exogenous gene can be a gene not native to and introduced as a transgene into the transgenic plant of the invention, or it can be a gene native to the transgenic plant of the invention but located in a context other than that in which it normally occurs in nature (e.g., a native gene operably linked to a non-native promoter and introduced as a transgene into the plant). The term “operably linked” when used in reference to the relationship between nucleic acid sequences and/or amino acid sequences refers to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest. The term also refers to the linkage of amino acid sequences in such a manner so that a functional protein is produced.

The exogenous gene encoding a polypeptide may be any exogenous gene of interest that is transcribed to an RNA transcript which is at least in part translatable to a polypeptide, preferably a gene that transcribes to a messenger RNA (mRNA) that contains or can be made to contain a 3′ untranslated region (3′ UTR) in which the destabilizing sequence can be placed. The exogenous gene may include a naturally occurring sequence or a derivative or homologue of such a naturally occurring sequence. Derivatives or homologues of naturally occurring sequences may include, but are not limited to, deletions of sequence, single or multiple point mutations, alterations at a particular restriction enzyme site, addition of functional elements, or other means of molecular modification of a naturally occurring sequence. Techniques for obtaining such derivatives are well known in the art. See, e.g., methodologies disclosed in Sambrook and Russell, 2001, incorporated by reference herein.

Non-limiting examples of suitable exogenous genes include genes encoding transcription factors and genes encoding enzymes involved in the biosynthesis or catabolism of molecules of interest (such as amino acids, fatty acids and other lipids, sugars and other carbohydrates, and biological polymers). Specific, non-limiting examples of suitable exogenous genes include genes encoding anthranilate synthase; genes involved in multi-step biosynthesis pathways, where it may be of interest to regulate the level of one or more intermediates, such as genes encoding enzymes for polyhydroxyalkanoate biosynthesis (see, e.g., U.S. Pat. No. 5,750,848, herein specifically incorporated by reference); genes encoding cell-cycle control proteins, such as proteins with cyclin-dependent kinase (CDK) inhibitor-like activity (see, e.g., genes disclosed in WO 05007829, herein specifically incorporated by reference); genes encoding proteins that, when expressed in transgenic plants, make the transgenic plants resistant to pests or pathogens (see, e.g., genes for cholesterol oxidase as disclosed in U.S. Pat. No. 5,763,245, herein specifically incorporated by reference); genes encoding proteins encoding a selectable trait (such as antibiotic resistance, especially if it is desirable to express such a gene at a level sufficient to permit selection of a cell carrying the gene but not so high as to allow adjacent cells not carrying the gene to “escape” or survive selection); genes where expression is preferably transient (e.g., genes involved in pest or pathogen resistance, especially when expression is pest- or pathogen-induced); and genes which can induce or restore fertility (see, e.g., the barstar/barnase genes described in U.S. Pat. No. 6,759,575, herein specifically incorporated by reference).

The destabilizing sequence can include any sequence that imparts instability to the exogenous gene's transcribed RNA, for example by decreasing stability or half-life of an mRNA transcribed from the endogenous gene. In one preferred embodiment, the destabilizing sequence is at least one selected from a 3′ SAUR terminator, a DST element, an ATTTA motif, an ATTTAA motif and a combination of ATTTA and ATTTAA motifs. The ATTTA or ATTTAA DNA motifs are transcribed to AUUUA or AUUUAA RNA motifs. Most preferably, presence of the destabilizing sequence results in expression of the exogenous gene at a lower level in the transgenic plant relative to expression in the absence of the destabilizing sequence. More than one destabilizing sequence, or multiple copies of one or more destabilizing sequences, may be used. In non-limiting examples, a transgenic plant of the invention may have in its genome DNA including an exogenous gene that has in its 3′ untranslated region at least one SAUR terminator, or multiple copies of DST elements, or a combination of SAUR terminators, DST elements, or a combination of ATTTA and ATTTAA motifs.

The 3′ SAUR terminator can be a 3′ SAUR terminator of known sequence, non-limiting examples of which include 3′ SAUR terminator variants amplified by PCR from Arabidopsis genomic DNA using primers based on a published SAUR gene, SAUR-AC1 (see Gil et al. (1994), which is incorporated by reference herein), and disclosed here as SEQ ID NO: 1, SEQ ID NO: 2, SEQ ID NO: 3, and SEQ ID NO: 4.

The 3′ SAUR terminator can also be a novel 3′ SAUR terminator homologue, which may be identified by one of ordinary skill in the art, for example, by identifying homologues to known SAUR genes and sequencing the 3′ UTR region of these genes, or by directly identifying homologues of known 3′ SAUR terminators.

Similarly, the DST element can be a known DST element or a novel DST element homologue. Non-limiting examples of a DST element include a DST element from soybean gene 15A, such as disclosed here as SEQ ID NO: 5 (“1× DST”), SEQ ID NO: 6 (“2× DST”), SEQ ID NO: 7 (“3× DST”), SEQ ID NO: 8 (“4× DST”), SEQ ID NO: 9 (“5× DST”), and SEQ ID NO: 10 (“6× DST”).

Suitable ATTTA motifs include sequences containing repeats of ATTTA. Suitable ATTTAA motifs include sequences containing repeats of ATTTAA. Preferably, at least 3 copies of the ATTTA or ATTTAA motifs are found in the repetitive sequence. Non-limiting embodiments include destabilizing sequences including 3, 4, 5, 6, 7, 8, 9, 10, 11, 12, 13, 14, 15, and even greater than 15 copies of the ATTTA or ATTTAA motifs in an overlapping repeat. Thus, non-limiting examples include 3× ATTTA (ATTTATTTATTTA (SEQ ID NO:27)), 5× ATTTA (ATTTATTTATTTATTTATTTA (SEQ ID NO:28)), 11× ATTTAA (ATTTAATTTAATTTAATTTAATTTAATTTAATTTAATTTAATTTAATTTAATTT AA (SEQ ID NO:29)), and a 7× ATTTA/ATTTAA combination (e.g., ATTTATTTATTTAATTTAATTTAATTTATTTAA (SEQ ID NO:30) and similar combinations). These examples are provided to illustrate the overlapping nature of the repeats and are not to be construed as limiting in any way.

Homologous sequences can be identified, e.g., by use of comparison tools known to those in the art, such as, but not limited to, BLAST (Altschul et al. (1997), which is incorporated by reference herein). Thus, genomic DNA sequences from a plant species of interest, especially a crop plant of interest, can be searched for SAUR homologues, homologues of known 3′ SAUR terminators, or homologous of known DST elements. One skilled in the art would realize that a variety of primers could be designed using known SAUR sequences, known 3′ SAUR terminator sequences, or known DST elements (e.g., sequences provided in Newman et al. (1993), McClure et al. (1989), and Yamamoto et al. (1992), and Gil et al. (1994), and sequences provided herein in SEQ ID NO: 1 through SEQ ID NO: 10) to amplify and isolate DNA for sequencing and assaying for the ability to destabilize mRNA transcripts using suitable methods such as those provided in this disclosure, thereby providing additional novel SAUR sequences, novel 3′ SAUR terminator sequences, or novel DST elements useful in the instant invention.

Furthermore, modifications to known or novel destabilizing sequences may be made by one versed in the art. Modifications may include, but are not limited to, deletions of sequence, single or multiple point mutations, alterations at a particular restriction enzyme site, addition of functional elements, repetition of elements, or other means of molecular modification which may leave unchanged, or even enhance, the destabilizing sequence's ability to destabilize mRNA transcripts. Techniques for obtaining such derivatives are well known in the art. See, for example, methodologies disclosed in Sambrook and Russell, 2001, incorporated by reference herein. Techniques for mutagenizing or creating deletions in a DNA segment are well known to those of skill in the art and are disclosed in detail, for example, in U.S. Pat. No. 6,583,338, which is incorporated herein by reference in its entirety.

In one non-limiting embodiment of the invention, the transgenic plant is a dicot crop plant (e.g., soybean) or a monocot crop plant (e.g., maize) wherein it is desired to provide a modified amino acid content in the transgenic crop plant or transgenic crop plant seed, and the exogenous gene is a gene for biosynthesis of an amino acid (e.g., lysine, tryptophan, or methionine); a destabilizing sequence or sequences can be used to express the amino acid biosynthesis gene at a lower level in the transgenic crop plant or seed relative to expression in the absence of the destabilizing sequence, thereby providing various options for the amino acid composition of the transgenic crop plant or seed.

The present invention also provides a transgenic plant having in its genome DNA including a non-constitutive promoter operably linked to an exogenous gene encoding a polypeptide and having in its 3′ untranslated region a destabilizing sequence, whereby the polypeptide is expressed at a lower level in the transgenic plant relative to expression in the absence of the destabilizing sequence.

The transgenic plant may be derived from any monocot or dicot plant of interest; in some preferred embodiments, the transgenic plant is a crop plant. A description of plants suited to the invention is provided above under the heading “Transgenic Plants I”.

Non-constitutive promoters suitable for use with the transgenic plants of the invention include spatially specific promoters, temporally specific promoters, and inducible promoters. Spatially specific promoters can include organelle-, cell-, tissue-, or organ-specific promoters (e.g., a plastid-specific, a root-specific, or a seed-specific promoter for suppressing expression of the target RNA in plastids, roots, or seeds, respectively). Temporally specific promoters can include promoters that tend to promote expression during certain developmental stages in a plant's growth cycle, or during different times of day or night, or at different seasons in a year. Inducible promoters include promoters induced by chemicals or by environmental conditions such as, but not limited to, biotic or abiotic stress (e.g., water deficit or drought, heat, cold, nutrient or salt levels, high or low light levels, or pest or pathogen infection). An expression-specific promoter can also include promoters that are generally constitutively expressed but at differing degrees or “strengths” of expression, including promoters commonly regarded as “strong promoters” or as “weak promoters”.

Many expression-specific promoters functional in plants and useful in the method of the invention are known in the art. For example, U.S. Pat. No. 5,837,848, U.S. Pat. No. 6,437,217, and U.S. Pat. No. 6,426,446 disclose root specific promoters; U.S. Pat. No. 6,433,252 discloses a maize L3 oleosin promoter; U. S. Patent Application Publication 2004/0216189 discloses a promoter for a plant nuclear gene encoding a plastid-localized aldolase; U.S. Pat. No. 6,084,089 discloses cold-inducible promoters; U.S. Pat. No. 6,140,078 discloses salt inducible promoters; U.S. Pat. No. 6,294,714 discloses light-inducible promoters; U.S. Pat. No. 6,252,138 discloses pathogen-inducible promoters; and U.S. Patent Application Publication 2004/0123347 discloses water deficit-inducible promoters. Each of the patents and publications disclosing promoters and their use, especially in recombinant DNA constructs functional in plants, are specifically incorporated herein by reference.

Nucleic acid sequences that are not naturally occurring promoters or promoter elements or homologues thereof but that can regulate expression of a gene may also be useful for use with the transgenic plants of the invention. Examples of such “gene independent” regulatory sequences include naturally occurring or artificially designed RNA sequences that include a ligand-binding region or aptamer and a regulatory region (which may be cis-acting). See, for example, Isaacs et al. (2004), Bayer and Smolke (2005), Mandal and Breaker (2004), Davidson and Ellington (2005), Winkler et al. (2002), Sudarsan et al. (2003), and Mandal and Breaker (2004), each of which is specifically incorporated by reference herein. Such “riboregulators” could be selected or designed for specific spatial or temporal specificity, for example, to regulate translation of the exogenous gene only in the presence (or absence) of a given concentration of the appropriate ligand.

The exogenous gene encoding a polypeptide may be any exogenous gene of interest that is transcribed to an RNA transcript which is at least in part translatable to a polypeptide, preferably a gene that transcribes to a messenger RNA (mRNA) that contains or can be made to contain a 3′ untranslated region (3′ UTR) in which the destabilizing sequence can be placed. Examples of suitable exogenous genes are described above under the heading “Transgenic Plants I”. The destabilizing sequence can include any sequence that imparts instability to the exogenous gene's transcribed RNA, for example by decreasing stability or half-life of an mRNA transcribed from the endogenous gene. In one preferred embodiment, the destabilizing sequence is at least one selected from a 3′ SAUR terminator, a DST element, an ATTTA motif, an ATTTAA motif and a combination of ATTTA and ATTTAA motifs, as also described above under the heading “Transgenic Plants I”.

The invention further provides a transgenic crop plant used for food or feed and having in its genome DNA including an exogenous gene encoding a polypeptide and having in its 3′ untranslated region a destabilizing sequence, whereby the polypeptide is expressed at a lower level in the transgenic crop plant used for food or feed relative to expression in the absence of the destabilizing sequence.

The transgenic crop plant used for food or feed can be any monocot or dicot crop plant used for food or feed, suitable examples of which are provided above under the heading “Transgenic Plants I”. Preferred dicot crop plants used for food or feed include, but are not limited to, canola, cotton, potato, quinoa, amaranth, buckwheat, safflower, soybean, sugarbeet, and sunflower, more preferably soybean, canola, and cotton. Preferred monocot crop plants used for food or feed include, but are not limited to, wheat, oat, barley, maize, rye, triticale, rice, forage grasses, sorghum, millet, and sugarcane, more preferably maize, wheat, and rice. In some specific embodiments, soybean and maize are particularly preferred plants.

The exogenous gene encoding a polypeptide may be any exogenous gene of interest that is transcribed to an RNA transcript which is at least in part translatable to a polypeptide, preferably a gene that transcribes to a messenger RNA (mRNA) that contains or can be made to contain a 3′ untranslated region (3′ UTR) in which the destabilizing sequence can be placed. Examples of suitable exogenous genes are described above under the heading “Transgenic Plants I”. The destabilizing sequence can include any sequence that imparts instability to the exogenous gene's transcribed RNA, for example by decreasing stability or half-life of an mRNA transcribed from the endogenous gene. In one preferred embodiment, the destabilizing sequence is at least one selected from a 3′ SAUR terminator, a DST element, an ATTTA motif, an ATTTAA motif and a combination of ATTTA and ATTTAA motifs, as also described above under the heading “Transgenic Plants I”.

### II. ATTTAA REPEATS

In another aspect, the present invention provides a transgenic plant having in its genome DNA comprising an exogenous gene encoding a polypeptide and having in its 3′ untranslated region a destabilizing sequence including overlapping ATTTAA repeats, whereby said polypeptide is expressed at a lower level in said transgenic plant relative to expression in the absence of said destabilizing sequence.

The transgenic plant may be derived from any monocot or dicot plant of interest; in some preferred embodiments, the transgenic plant is a crop plant. A description of plants suited to this aspect of the invention is provided above under the heading “Transgenic Plants I”.

The exogenous gene encoding a polypeptide may be any exogenous gene of interest that is transcribed to an RNA transcript which is at least in part translatable to a polypeptide, preferably a gene that transcribes to a messenger RNA (mRNA) that contains or can be made to contain a 3′ untranslated region (3′ UTR) in which the destabilizing sequence can be placed. Examples of suitable exogenous genes are described above under the heading “Transgenic Plants I”.

The destabilizing sequence includes overlapping ATTTAA repeats. In one preferred embodiment, the destabilizing sequence includes at least 3 overlapping ATTTAA repeats, and can include 4, 5, 6, 7, 8, 9, 10, 11, 12, 13, 14, 15, and even greater than 15 copies of the ATTTAA motif in an overlapping repeat. Thus, non-limiting examples include 3× ATTTAA (ATTTAATTTAATTTAA; SEQ ID NO:31), 5× ATTTAA (ATTTAATTTAATTTAATTTAATTTAA; SEQ ID NO:32), and 11× ATTTAA (ATTTAATTTAATTTAATTTAATTTAATTTAATTTAATTTAATTTAATTTAATTT AA; SEQ ID NO:29). In other embodiments, the overlapping ATTTAA repeats can be found in combination with ATTTA repeats, such as in the combinations described above under the heading “Transgenic Plants I”.

### III. TRANSGENIC PLANTS WITH MODERATED ANTHRANILATE SYNTHASE EXPRESSION

The present invention further provides a transgenic plant having in its genome DNA including a gene encoding anthranilate synthase and having in its 3′ untranslated region a destabilizing sequence, whereby the anthranilate synthase is expressed at a lower level in the transgenic plant relative to expression in the absence of the destabilizing sequence.

The transgenic plant may be derived from any monocot or dicot plants of interest such as are provided above under the heading “Transgenic Plants I”. In some preferred embodiments, the transgenic plant may be a crop plant, such as crop plants wherein it is desired to increase the levels of tryptophan in the entire plant or in specific plant tissues or cells. Non-limiting examples include embodiments where the transgenic plant is soybean or corn.

The gene encoding anthranilate synthase may be any naturally occurring gene for anthranilate sequence, or homologues of these genes, such as may be identified from sequence databases by use of comparison tools known to those in the art, such as, but not limited to, BLAST (Altschul et al. (1997), which is incorporated by reference herein). The gene encoding anthranilate synthase may include a derivative sequence based on a naturally occurring anthranilate synthase but with one or more modifications such as deletions of sequence, single or multiple point mutations, alterations at a particular restriction enzyme site, addition of functional elements, repetition of elements, or other means of molecular modification. Such modifications may be made to enhance or alter the anthranilate synthase's properties in the transgenic plant. A non-limiting example of modification includes codon optimization of a prokaryotic anthranilate synthase for expression in a transgenic plant.

The gene for anthranilate synthase preferably contains in its 3′ untranslated region a destabilizing sequence, which can include any sequence that imparts instability to the gene for anthranilate synthase's transcribed RNA, for example by decreasing stability or half-life of an mRNA transcribed from the gene for anthranilate synthase. In one preferred embodiment, the destabilizing sequence is at least one selected from a 3′ SAUR terminator, a DST element, an ATTTA motif, an ATTTAA motif and a combination of ATTTA and ATTTAA motifs, as also described above under the heading “Transgenic Plants I”. Most preferably, the destabilizing sequence is such that the anthranilate synthase is expressed at a lower level in the transgenic plant relative to expression in the absence of the destabilizing sequence.

### IV. PROVIDING TRANSGENIC PLANTS

Various aspects of the present invention are directed to transgenic plants as described in the preceding paragraphs. The present invention contemplates and claims transgenic plants (in many embodiments transgenic crop plants in particular), both directly regenerated from cells which have been transformed with transgenic DNA including an exogenous gene that has in its 3′ untranslated region a destabilizing sequence, as well as progeny of such transgenic plants, for example, inbred progeny and hybrid progeny of transformed plants.

Preparation of nucleic acid constructs for transformation of plant cells and production of the transgenic plant make use of techniques well known in the art. See, for example, methodologies disclosed in Maliga et al., 1995, and Sambrook and Russell, 2001, which are specifically incorporated by reference herein. One of ordinary skill in the art would be familiar with techniques for transforming plant cells to provide a transgenic plant of the invention. See, for example, microprojectile bombardment methods as disclosed in U.S. Pat. Nos. 5,550,318, 5,538,880, 6,160,208, and 6,399,861, and Agrobacterium-mediated transformation methods as described in U.S. Pat. No. 5,591,616, each of which is herein specifically incorporated by reference. Useful techniques for transforming plant cells using site-specific integration include the cre-lox system disclosed in U.S. Pat. No. 4,959,317 and the FLP-FRT system disclosed in U.S. Pat. No. 5,527,695, both of which are incorporated by reference herein.

Transformation of plant cells to yield transgenic plants of the invention is preferably practiced in tissue culture on media and in a controlled environment. Practical transformation methods and materials for making transgenic plants of this invention, e.g., various media and recipient target cells, transformation of immature embryos, and subsequent regeneration of fertile transgenic plants, are disclosed in U.S. Pat. Nos. 6,194,636 and 6,232,526, which are incorporated herein by reference.

After delivery of the transgenic DNA to recipient plant cells, transformed cells are generally identified for further culturing and plant regeneration. To improve the ability to identify transformants, one may employ a selectable or screenable marker gene, where the potentially transformed cell population can be assayed by exposing the cells to a selective agent or agents or screened for the desired marker gene trait. Non-limiting examples of screenable markers include a gene expressing a colored or fluorescent protein such as a luciferase or green fluorescent protein (GFP), or a gene expressing a beta-glucuronidase or uidA gene (GUS) for which various chromogenic substrates are known. Non-limiting examples of selectable markers include those conferring resistance to antibiotics such as kanamycin and paromomycin (nptII), hygromycin B (aph IV) and gentamycin (aac3 and aacC4) or resistance to herbicides such as glufosinate (bar or pat) and glyphosate (aroA or EPSPS); particularly useful examples of such selectable markers are illustrated in U.S. Pat. Nos. 5,550,318, 5,633,435, 5,780,708, and 6,118,047, all of which are specifically incorporated by reference herein.

The transgenic plants of the present invention may be further modified or hybridized to provide derivative transgenic plants having stacked traits, such as additional agronomically desirable traits, the techniques for which are known to one of ordinary skill in the art. See, e.g., U.S. Patent Application Publications 2003/0106096 and 2002/0112260, and U.S. Pat. Nos. 5,034,322, 5,776,760, 6,107,549, and 6,376,754, all of which are specifically incorporated herein by reference. Non-limiting examples of such traits include, but are not limited to, resistance or tolerance of abiotic stress such as drought or temperature stress, and resistance to pests or pathogens as illustrated by U.S. Pat. Nos. 5,250,515, 5,880,275, 6,506,599, and 5,986,175, and U.S. Patent Application Publication 2003/0150017 A1, all of which are incorporated herein by reference. Seeds of transgenic plants can be harvested from fertile transgenic plants and be used to grow progeny generations of transformed plants of this invention including hybrid plant lines useful, for example, for screening of plants having an enhanced agronomic trait. In addition to direct transformation of a plant with a recombinant DNA, transgenic plants can be prepared by crossing a first plant having a recombinant DNA with a second plant lacking the DNA. For example, recombinant DNA can be introduced into a first plant line that is amenable to transformation to produce a transgenic plant which can be crossed with a second plant line to introgress the recombinant DNA into the second plant line. A transgenic plant with recombinant DNA providing an enhanced agronomic trait, e.g., enhanced yield, can be crossed with transgenic plant line having other recombinant DNA that confers yet another trait, e.g., herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA. Progeny plants carrying DNA for both parental traits can be crossed back into the female parent line multiple times, e.g., usually 6 to 8 generations, to produce a progeny plant with substantially the same genotype as one original transgenic parental line but for the recombinant DNA of the other transgenic parental line.

### V. POST-TRANSCRIPTIONAL REGULATION OF GENE EXPRESSION BY CONTROLLING MESSAGE STABILITY

The present invention also provides a method to post-transcriptionally decrease message stability of a gene of interest in a crop plant used for food or feed, including adding a destabilizing sequence to the 3′ untranslated region of the gene of interest in the crop plant used for food or feed, whereby message stability of the gene of interest is post-transcriptionally decreased. Preferably, the post-transcriptional decrease of message stability results in expression of the gene at a level lower than that where the destabilizing sequence is absent.

The transgenic crop plant used for food or feed can be any monocot or dicot crop plant used for food or feed, suitable examples of which are provided above under the heading “Transgenic Plants I”. Preferred dicot crop plants used for food or feed include, but are not limited to, canola, cotton, potato, quinoa, amaranth, buckwheat, safflower, soybean, sugarbeet, and sunflower, more preferably soybean, canola, and cotton. Preferred monocot crop plants used for food or feed include, but are not limited to, wheat, oat, barley, maize, rye, triticale, rice, forage grasses, sorghum, millet, and sugarcane, more preferably maize, wheat, and rice. In some specific embodiments, soybean and maize are particularly preferred plants.

The gene of interest can be any gene that can be post-transcriptionally regulated by means of a destabilizing sequence, thus, any gene of interest that is transcribed to an RNA transcript which is at least in part translatable to a polypeptide, preferably a gene that transcribes to a messenger RNA (mRNA) that contains or can be made to contain a 3′ untranslated region (3′ UTR) in which the destabilizing sequence can be placed. Examples of suitable genes of interest are the exogenous genes as described above under the heading “Transgenic Plants I”. Non-limiting examples of suitable genes of interest include genes involved in the biosynthesis of molecules of interest, such as amino acids, fatty acids and other lipids, and sugars and other carbohydrates. In one embodiment of the invention, the gene of interest is operably linked to at least one promoter element in a transgenic expression cassette.

The destabilizing sequence can include any sequence that imparts instability to the gene of interest's transcribed RNA, as described above under the heading “Transgenic Plants I”. In one preferred embodiment, the destabilizing sequence is at least one selected from a 3′ SAUR terminator, a DST element, an ATTTA motif, an ATTTAA motif, and a combination of ATTTA and ATTTAA motifs. The ATTTA or ATTTAA DNA motifs are transcribed to AUUUA or AUUUAA RNA motifs. Most preferably, presence of the destabilizing sequence results in expression of the gene of interest at a lower level in the transgenic plant relative to expression in the absence of the destabilizing sequence. More than one destabilizing sequence, or multiple copies of one or more destabilizing sequences, may be used. In non-limiting examples, a transgenic plant of the invention may have in its genome DNA including a gene of interest that has in its 3′ untranslated region at least one SAUR terminators, or multiple copies of DST elements, or any combination of SAUR terminators, DST elements, or AUUUA or AUUUAA motifs.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All nucleic acid sequences are given in the 5′ to 3′ direction unless otherwise stated. The constructs and vectors described herein are provided as illustrative examples and are not to be taken as limiting in any manner.

### Example 1

### Cloning of SAUR Terminators From Arabidopsis

This example illustrates destabilizing sequences useful in the present invention. More specifically, this example describes cloning of SAUR terminators from Arabidopsis.

The terminator of the Arabidopsis thaliana SAUR-AC1 gene (Gil et al., 1994), which is incorporated by reference in its entirety herein) was PCR amplified from Arabidopsis (cv. Columbia) genomic DNA using primers SAURfor1, ACCAGCCTTTGTTTCAACAA (SEQ ID NO:11), and SAURrev1, CATAATCAATAAGAAAATAGATGTAC (SEQ ID NO:12) designed according to the published gene sequence and supplied by Invitrogen (Carlsbad, Calif.).

PCR was performed with the Expand High Fidelity PCR System (catalogue number 1 732 641, Roche Molecular Biochemicals, Indianapolis, Ind.). The primary PCR components and conditions were as given in Table 1.

TABLE 1

| | |
|---|---|
| Component | Amount |

Primary PCR (see Example 1)

| | | |
|---|---|---|
| dNTP mix (10 millimolar of each dNTP) | 1.0 | microliter |
| Primer SAURfor1 (SEQ ID NO: 11) | 1.0 | microliter |
| (100 micromolar) | | |
| Primer SAURrev1 (SEQ ID NO: 12) | 1.0 | microliter |
| (100 micromolar) | | |
| 10X PCR Buffer (containing MgCl₂) | 5 | microliters |
| Enzyme mix | 1 | microliter |
| Arabidopsis Genomic DNA (Template) | 0.1 | microgram DNA |
| H₂O | to 50 | microliters |
| | | final volume |

Nested PCR (see Example 1)

| | | |
|---|---|---|
| dNTP mix (10 millimolar of each dNTP) | 1.0 | microliter |
| Primer SAURfor2EcoRI (SEQ ID NO: 13) | 1.0 | microliter |
| 100 micromolar) | | |
| Primer SAURrev2NotI (SEQ ID NO: 14) | 1.0 | microliter |
| (100 micromolar) | | |
| 10X PCR Buffer (containing MgCl₂) | 5 | microliters |
| Enzyme mix | 1 | microliter |
| Purified primary PCR product (Template) | 5 | microliters |
| H₂O | to 50 | microliters |
| | | final volume |

Fragment Assembly PCR (see Example 3)

| | | |
|---|---|---|
| dNTP mix (10 millimolar of each dNTP) | 1.0 | microliter |
| Primer 2XDSTfor (SEQ ID NO: 15) | 1.0 | microliter |
| 100 micromolar) | | |
| Primer 2XDSTrev (SEQ ID NO: 16) | 1.0 | microliter |
| (100 micromolar) | | |
| 10X PCR Buffer (containing MgCl₂) | 5 | microliters |
| Enzyme mix | 1 | microliter |
| Purified primary PCR product (Template) | 5 | microliters |
| H₂O | to 50 | microliters |
| | | final volume |

Fragment Assembly PCR (see Example 7)

| | | |
|---|---|---|
| Forward anneal 1XDST (SEQ ID NO: 17) | 2.0 | microliter |
| 100 micromolar) | | |
| Rev Comp anneal 1XDST (SEQ ID NO: 18) | 2.0 | microliter |
| (100 micromolar) | | |
| 10X PCR Buffer (containing MgCl₂) | 5 | microliters |
| Enzyme mix | 1 | microliter |
| Purified primary PCR product (Template) | 5 | microliters |
| H₂O | to 50 | microliters |
| | | final volume |

After the reaction was initiated by denaturing the sample at 94 degrees Celsius for 1 minute, the reaction mixture was incubated for 20 cycles consisting of 94 degrees Celsius for 15 seconds, 68 degrees Celsius for 30 seconds (decreased 1 degree Celsius per cycle) and 72 degrees Celsius for 3 minutes. The reaction mixture was then incubated for 11 cycles consisting of 94 degrees Celsius for 15 seconds, 48 degrees Celsius for 30 seconds, 72 degrees Celsius for 3 minutes. The process was concluded with a step of 72 degrees Celsius for 10 minutes and the reaction mixture was held at 4 degrees Celsius until next experiment.

Products from the primary PCR reaction were purified using QIAquick PCR purification kit (catalogue number 28104, QIAGEN Inc., Valencia, Calif.) and eluted in 30 microliters H₂O. A second reaction using nested PCR primers was performed using 5 microliters of purified PCR product from the primary reaction as template and primers SAURfor2EcoRI, AAAGAATTCAACTAGTAGGATCCAGTACTATACTACAACATTTCC (SEQ ID NO:13), and SAURrev2Not, AAAGCGGCCGCCCGGGACCGGACTAACCGCAGTTCA (SEQ ID NO:14) designed according to the published gene sequence and supplied by Invitrogen (Carlsbad, Calif.).

The PCR was performed with the Expand High Fidelity PCR System (catalogue number 1 732 641, Roche Molecular Biochemicals, Indianapolis, Ind.). The nested PCR components and conditions were as given in Table 1; the amplification reaction was carried out as described above.

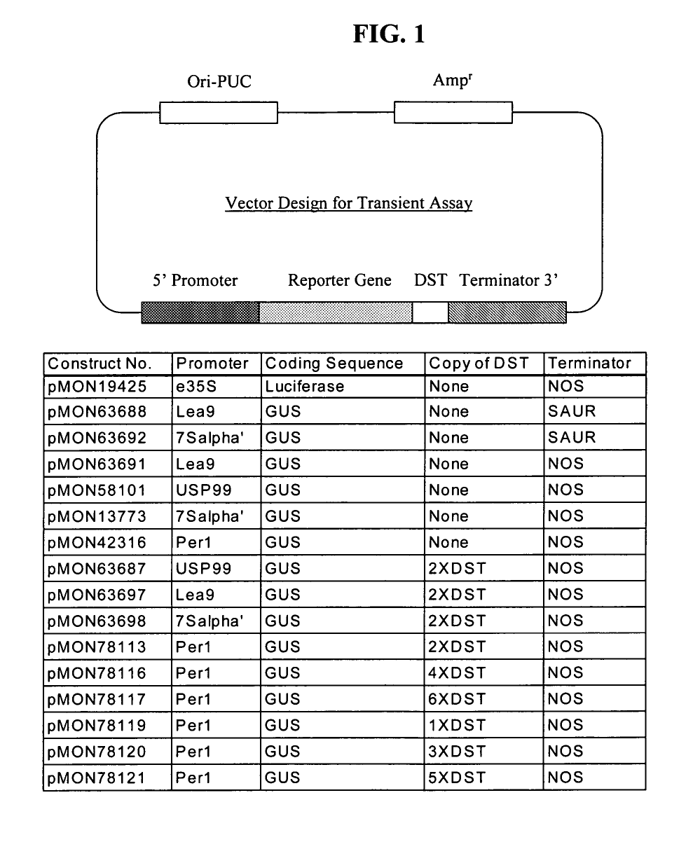

Product of the nested PCR product was cleaned using QIAquick PCR Purification Kit (catalogue number 28104, QIAGEN Inc., Valencia, Calif.) and eluted in 30 microliters ddH₂O. An aliquot of 5 microliters eluted DNA was digested with NotI and EcoRI. The digested product was separated in agarose gel. The band of expected size (˜750 bp) was excised and purified using the QIAquick Gel Extraction Kit (catalogue number 28704, QIAGEN Inc., Valencia, Calif.). The putative SAUR fragments were cloned as 3′terminators into a PUC plasmid that contained Lea9 as promoter and GUS as coding sequence. The resulting construct (pMON63688) is depicted in FIG. 1. The multiple clones of pMON63688 constructs were sequenced and several variants of SAUR terminators were identified based on sequence comparison (Dnastar software package, DNASTAR, Inc., Madison, W153715; www.dnastar.com). In a separate cloning experiment, NOS terminator was cloned into the same backbone vector with Lea9 promoter and GUS coding sequence to provide pMON63691 (FIG. 1), which was used as a control in transient assays comparing the effects of the SAUR terminator with those of the NOS terminator.

Another construct, pMON13773 (FIG. 1), was made to contain 7Salpha′ promoter driving GUS with NOS terminator. The variant 1 of SAUR was cloned into pMON13773 to replace NOS terminator. The new construct, pMON63692, contained 7Salpha′ promoter driving GUS with NOS terminator (FIG. 1).

### Example 2

### Characterization of SAUR Terminators in a Soybean Transient Transformation System

This example illustrates destabilizing sequences useful in the present invention and their use in a transgenic plant model. More specifically, this example describes characterization of SAUR terminators in a soybean transient transformation system.

Seeds from a dicot crop plant, soybean (Asgrow A3244), were harvested 25-28 days after flowering and osmotically treated overnight at 25 degrees Celsius in the dark on GAMBORG's medium (catalogue number G5893, Sigma Company, St. Louis, Mo.) supplemented with 50 millimolar glutamine, 111 millimolar maltose, 125 millimolar raffinose, 125 millimolar mannitol and 3 grams/liter purified agar, pH 5.6. The resulting cotyledons were separated and bombarded with purified supercoiled DNA of pMON63691 (NOS terminator) or pMON63688 (SAUR terminators) using particle gun technology (Maliga et al., 1995). As an internal control to normalize experimental variation, a separate e35S-driven luciferase construct pMON19425 (FIG. 1) was included at a concentration of 1 microgram/microliter and in a 1:1 molar ratio with each of the test constructs. Each plate had six cotyledons and 5 to 6 replicate plates were bombarded per construct. Bombarded tissues were incubated for 48 hours at 25 degrees Celsius.

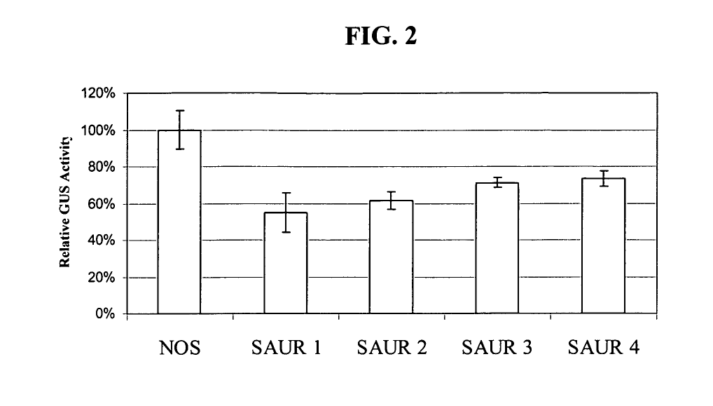

Proteins were extracted from six bombarded soybean cotyledons using 1 milliliter extraction buffer containing 0.1 molar potassium phosphate (pH 7.8), 10 millimolar DTT, 1 millimolar EDTA, 5% glycerol, and proteinase inhibitor (1 tablet/50 milliliters, catalogue number 1 697 498, Roche Molecular Biochemicals, Indianapolis, Ind.). A 100-microliter aliquot of the protein extract was used for a luciferase assay following a “Steady-Glo” procedure by Promega (catalogue number E2510, Promega Corporation Madison, Wis.). GUS assay buffer was made by adding 8.8 milligrams MUG (4-methylumbelliferyl beta-D-glucuronide, catalogue number M9130, Sigma, St. Louis, Mo.) to 10 milliliters extraction buffer. A 50-microliter aliquot of the protein extract was mixed with 200 microliters of GUS assay buffer. GUS assay was performed on a Spectramax Gemini spectrophotometric plate reader using the Basic Kinetic Protocol of Softmax Pro software with 355 nanometer excitation, 460 nanometer emission, and 455 nanometer cutoff (Molecular Devices, Sunnyvale, Calif.). Fluorescence readings were recorded over a period of 2 hours with 7 minutes intervals and a GUS Vₘₐₓ was obtained for each sample. Each sample was assayed twice and the average value was used for data analysis. GUS activity was normalized according to each sample's luciferase activity and the relative promoter strength was expressed by setting the control vector pMON63691 (FIG. 1) arbitrarily to 100%. Alternatively, GUS proteins purified from transgenic plant (catalogue number G8162, Sigma, St. Louis, Mo.) at known concentrations were included in the assay for the calculation of absolute amount of GUS in the samples. The results (FIG. 2) indicated that all the variants of the SAUR terminators significantly decreased the expression of GUS when compared to NOS terminator, a benchmark terminator for gene expression in transgenic plants.

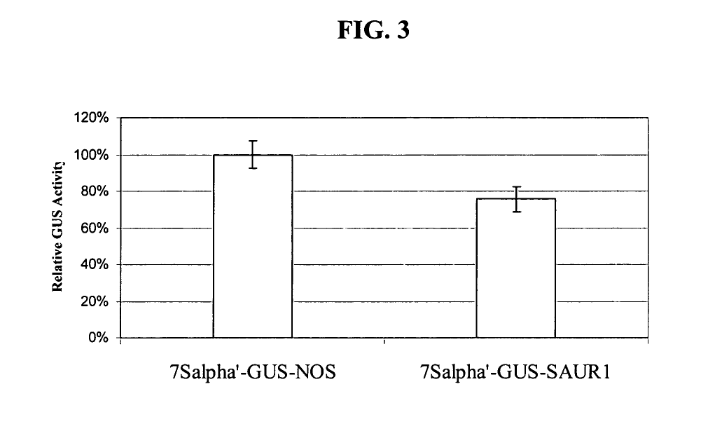

Another transient assay was done in a similar fashion to show that SAUR terminators effectively decreased gene expression when 7Salpha′ was used as a promoter to drive GUS expression (FIG. 3).

### Example 3

### Cloning of Multiple Copies of DST Elements into Vectors With Various Promoters

This example further illustrates destabilizing sequences useful in the present invention, and their use with a gene of interest operably linked to at least one promoter element in a transgenic expression cassette. More specifically, this example describes cloning of multiple copies of DST elements into vectors with various promoters.

The DST element in SAUR genes was identified as a key element responsible for destabilizing mRNA. To test if the DST element works effectively in conjunction with heterologous expression cassette in soybean, two single-stranded oligonucleotide fragments, 2× DSTfor, AAAGAATTCGCTAGCAGGAGACTGACATAGATTGGAGGAGACATTTTGTATA ATAAGGAGACTGACATAG (SEQ ID NO: 15), and 2× DSTrev, AAAGGATCCGATGGCCGCACTAGTTATTATACAAAATGTCTCCTCCAATCTAT GTCAGTCTCCTTATTAT (SEQ ID NO:16) were designed for assembly of a double stranded DNA fragment containing two copies of DST, and supplied by Invitrogen (Carlsbad, Calif.).

PCR for fragment assembly was performed with the Expand High Fidelity PCR System (catalogue number 1 732 641, Roche Molecular Biochemicals, Indianapolis, Ind.). Because the two single-stranded DNA fragments have over-lapping regions that are complementary to each other, template DNA was not needed. PCR for fragment assembly components and conditions were as given in Table 1; the reaction was carried out as described in Example 1.

A 3-microliter aliquot of the PCR reaction was digested with EcoRI and BamHI enzymes and cloned into pMON58101 (FIG. 1) that was linearized at sites between GUS coding gene and NOS terminator. Clones containing 2× DST were identified by sequencing and named pMON63687 (FIG. 1). Comparison between pMON58101 and pMON63687 was expected to demonstrate the effect of 2× DST on gene expression. Two additional vectors were made by replacing the USP promoter in pMON63687 with a Lea9 promoter or a 7Salpha′ promoter to generate pMON63697 and pMON63698 (FIG. 1). pMON63697 was compared with pMON63691, a control vector contains 7Salpha′ promoter driving GUS with NOS terminator. pMON63698 was compared with pMON1 3773, another control vector contains 7Salpha′ promoter driving GUS with NOS terminator. The comparisons used a transient assay and are described in Example 4.

### Example 4

### Characterization of 2× DST in a Soybean Transient Transformation System

This example illustrates destabilizing sequences useful in the present invention, and their use with a gene of interest operably linked to at least one promoter element in a transgenic expression cassette. More specifically, this example describes characterization of 2× DST in a soybean transient transformation system.

Seeds from soybean plants (Asgrow A3244) were harvested 25-28 days after flowering and osmotically treated as described in Example 2. The resulting cotyledons were separated and bombarded with purified supercoiled DNA of pMON58101 (NOS terminator) or pMON63687 (2× DST and NOS terminator) using particle gun technology as described in Example 2. The control vector was an e35S-driven luciferase construct, pMON19425.

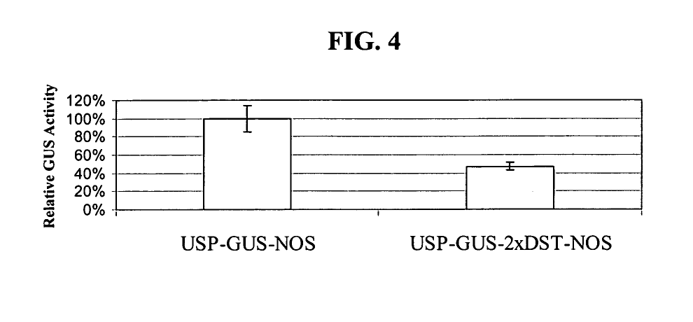

Proteins were extracted and analysed with a “Steady-Glo” luciferase assay and a GUS assay as described in Example 2. GUS activity was normalized according to each sample's luciferase activity and the relative promoter strength was expressed by setting the control vector pMON58101 (FIG. 1) arbitrarily to 100%. The results (FIG. 4) indicated that inclusion of 2× DST significantly decreased the expression of GUS when compared to NOS terminator alone.

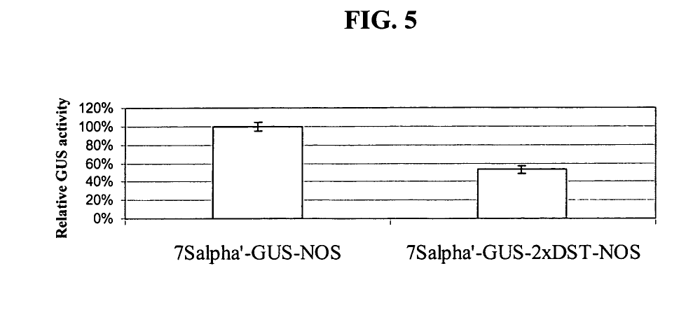

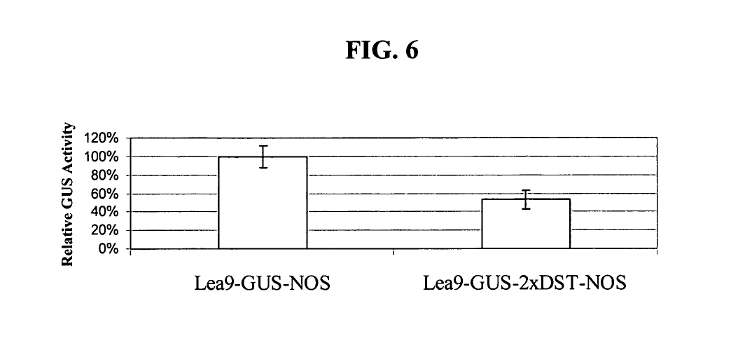

Other experiments were carried out to compare pMON13773 and pMON63698 (FIG. 5) and to compare pMON63691 and pMON63697 (FIG. 6). The results consistently showed that 2× DST effectively decreased gene expression in soybean cotyledons. The data collectively showed that 2× DST can work effectively regardless of promoters used in the experiment.

### Example 5

### Cloning of Per1 Vectors Containing 2× DST, 4× DST or 6× DST

This example illustrates destabilizing sequences useful in the present invention, and their use with a gene of interest operably linked to at least one promoter element in a transgenic expression cassette. More specifically, this example describes cloning of Per1 vectors containing 2× DST, 4× DST or 6× DST.

Additional vectors were constructed by following standard molecular cloning protocols (Sambrook and Russell, 2001, which is incorporated herein by reference) to test the effect of DST copy number on the level of gene expression. Vector pMON42316 (FIG. 1), the control vector, contained a Per1 promoter driving GUS expression with a NOS terminator. The Lea9 promoter in pMON63697 was excised and replaced with Per1 promoter to make pMON78113 which has 2× DST in combination with NOS terminator (FIG. 1).

To make multiple copies of DST, a 5-microliter aliquot of the PCR product from Example 3 was digested with SpeI and NheI. The digested DNA was separated on an agarose gel, extracted using QIAquick Gel Extraction Kit (catalogue number 28704, QIAGEN Inc., Valencia, Calif.) and ligated into pMON78113 that was linearized with SpeI and treated with CIP alkaline phosphatase. Clones containing 4× DST were selected by SpeI and NheI double digestion and named pMON78116 (FIG. 1).

pMON78116 was again linearized with SpeI, treated with CIP alkaline phosphatase and ligated to the 2× DST fragment prepared earlier. Clones containing 6× DST were selected by SpeI and NheI double digestion and named pMON78117 (FIG. 1).

### Example 6

### Comparison of 2× DST, 4× DST or 6× DST in a Soybean Transient Transformation System

This example illustrates destabilizing sequences useful in the present invention, and their use with a gene of interest operably linked to at least one promoter element in a transgenic expression cassette. More specifically, this example describes comparison of 2× DST, 4× DST and 6× DST in a soybean transient transformation system.

Seeds from soybean plants (Asgrow A3244) were harvested 25-28 days after flowering and osmotically treated as described in Example 2. The resulting cotyledons were separated and bombarded with purified supercoiled DNA of pMON42316 (NOS terminator only), pMON78113 (2× DST and NOS terminator), pMON78116 (4× DST and NOS terminator) or pMON78117 (6× DST and NOS terminator) using particle gun technology as described in Example 2. The control vector was an e35S-driven luciferase construct, pMON 19425.

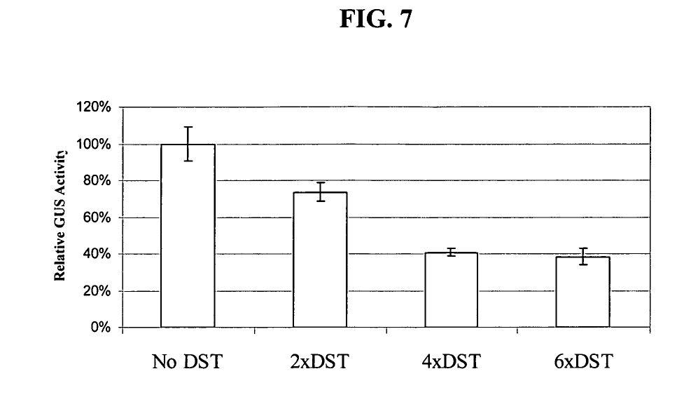

Proteins were extracted and analysed with a “Steady-Glo” luciferase assay and a GUS assay as described in Example 2. GUS activity was normalized according to each sample's luciferase activity and the relative promoter strength was expressed by setting the control vector pMON42316 (FIG. 1) arbitrarily to 100%. The results (FIG. 7) indicated that DST copy number negatively correlate with the level of gene expression.

### Example 7

### Cloning of 1× DST, 3× DST and 5× DST

This example illustrates destabilizing sequences useful in the present invention. More specifically, this example describes cloning of 1× DST, 3× DST and 5× DST.

Additional vectors are made to further evaluate the correlation of DST copy number and gene expression level. Two single stranded oligonucleotide fragments, Forward anneal 1× DST, CTAGCTAGGAGACTGACATAGATTGGAGGAGACATTTTGTATAATAGGA (SEQ ID NO:17), and Rev Comp anneal 1× DST, CTAGTCCTATTATACAAAATGTCTCCTCCAATCTATGTCAGTCTCCTAG (SEQ ID NO:18) were designed for assembly of a double stranded DNA fragment containing one copy of DST, and supplied by Integrated DNA Technologies, Inc. (Coralville, Ill.). Annealing of the two fragments was performed in 1× PCR buffer supplied in the Expand High Fidelity PCR System (catalogue number 1 732 641, Roche Molecular Biochemicals, Indianapolis, Ind.).

After the reaction was initiated by denaturing the sample at 96 degrees Celsius for 10 minutes, the temperature was decreased at a speed of 0.2 degrees Celsius/second and paused for 7 minutes each at 80 degrees Celsius, 70 degrees Celsius, 60 degrees Celsius, 50 degrees Celsius, 40 degrees Celsius and 30 degrees Celsius. The process ended at 4 degrees Celsius and the mixture was stored at 4 degrees Celsius until the next experiment.

The annealed 1× DST fragment was then purified using QIAquick PCR Purification Kit (catalogue number 28104, QIAGEN Inc., Valencia, Calif.) and eluted in 32 microliters double-distilled H₂O. The eluted DNA was treated using a T4 Polynucleotide Kinase Kit (catalogue number 18004-010, Invitrogen, Carlsbad, Calif.) and saved as 1× DST insert. To create 1× DST construct, pMON78113 was digested with SpeI and NheI and treated with CIP alkaline phosphatase. The backbone was then ligated to 1× DST insert to create pMON78119 (FIG. 1).

To create 3× DST and 5× DST constructs, pMON78113 and pMON78116 were linearized using SpeI and treated with CIP alkaline phosphatase. The backbone was then ligated to 1× DST insert. Clones containing 3× DST or 5× DST were selected by SpeI and NheI double digestion and named pMON78120 and pMON78121 respectively (FIG. 1).

### Example 8

### Comparison of 1× DST, 2× DST, 3× DST, 4× DST and 5× DST in a Soybean Transient Transformation System

This example illustrates destabilizing sequences useful in the present invention, and their use with a gene of interest operably linked to at least one promoter element in a transgenic expression cassette. More specifically, this example describes comparison of 1× DST, 2× DST, 3× DST, 4× DST and 5× DST in a soybean transient transformation system.

Seeds from soybean plants (Asgrow A3244) were harvested 25-28 days after flowering and osmotically treated as described in Example 2. The resulting cotyledons were separated and bombarded with purified supercoiled DNA of pMON42316 (NOS terminator only), pMON78119(1× DST and NOS terminator), pMON78113 (2× DST and NOS terminator), pMON78120(3× DST and NOS terminator), pMON78116 (4× DST and NOS terminator) or pMON78121 (5× DST and NOS terminator) using particle gun technology as described in Example 2. The control vector was an e35S-driven luciferase construct, pMON19425.

Proteins were extracted and analysed with a “Steady-Glo” luciferase assay and a GUS assay as described in Example 2. GUS activity was normalized according to each sample's luciferase activity and the relative promoter strength was expressed by setting the control vector pMON42316 (FIG. 1) arbitrarily to 100%. The results (FIG. 7) indicated that DST copy number was negatively correlated with the level of gene expression. The results also showed that 1× DST was sufficient to decrease gene expression.

### Example 9

### Effectiveness of Destabilizing Sequences in a Transgenic Crop Plant (Soybean)

This example illustrates destabilizing sequences useful in the present invention, and their use with a gene of interest operably linked to at least one promoter element in a transgenic expression cassette. More specifically, this example demonstrates effectiveness of destabilizing sequences in a transgenic crop plant (soybean).

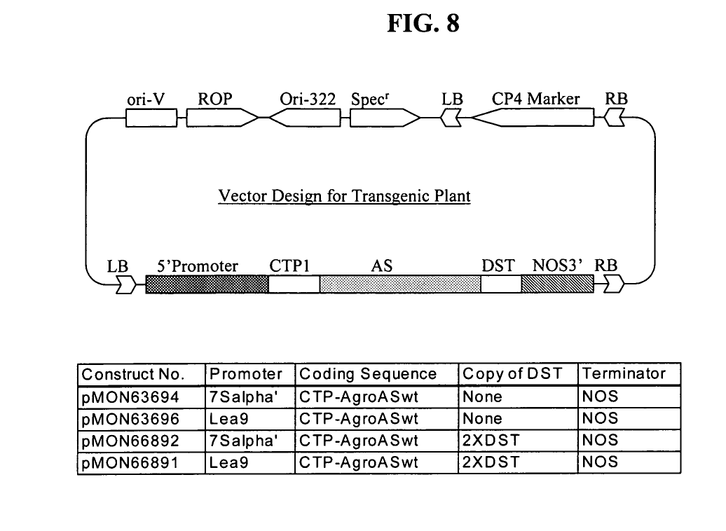

To further confirm the effectiveness of DST as a message-destabilizing sequence in transgenic soybean, multiple Agrobacterium transformation vectors (FIG. 8) were constructed by following standard molecular cloning protocols (Sambrook and Russell, 2001, which is incorporated herein by reference). An expression cassette consisting of FMV promoter, CTP2 and CP4 coding gene and E9 3′UTR was included as a selectable marker in all vectors. A fusion protein of Arabidopsis SSU IA CTP and Agrobacterium anthranilate synthase (AS) was used as a coding gene to deregulate the tryptophan biosynthetic pathway. Per1, Lea9, or 7Salpha′ promoter was used to drive the expression of the AS gene. NOS terminator was used in combination with DST at the 3′ end of the AS cassette.

The vectors described above were transferred into Agrobacterium tumefaciens, strain ABI by a triparental mating method (Ditta et al. (1980), which is incorporated by reference herein). The bacterial cells were prepared for transformation by methods well known in the art.

Commercially available soybean seeds (Asgrow A3244) were germinated over a 10-12 hour period. The meristem explants were excised and placed in a wounding vessel and wounded by sonication. Following wounding, the Agrobacterium culture described above was added and the explants were incubated for approximately one hour. Following inoculation, the Agrobacterium culture was removed by pipetting and the explants placed in co-culture for 2-4 days. The explants were then transferred to selection media consisting of Woody Plant Medium (WPM) (see McCown & Lloyd (1981), which is incorporated by reference in its entirety herein), plus 75 micromolar glyphosate and antibiotics to control Agrobacterium overgrowth, for 5-7 weeks to allow selection and growth of transgenic shoots. Phenotype-positive shoots were harvested approximately 5-7 weeks post inoculation and placed into selective rooting media comprising Bean Rooting Media (BRM) with 25 micromolar glyphosate (see U.S. Pat. No. 5,914,451, which is incorporated by reference in its entirety herein) for 2-3 weeks. Shoots producing roots were transferred to the greenhouse and potted in soil. Shoots that remained healthy on selection, but did not produce roots, were transferred to non-selective rooting media (i.e., BRM without glyphosate) for an additional two weeks. Tissues from any shoots that produced roots off selection were tested for expression of the plant selectable marker before they were transferred to the greenhouse and potted in soil. Plants were maintained under standard greenhouse conditions until R1 seed harvest.

The levels of free amino acids were analyzed from each of the transgenic events using the following procedure. Seeds from each of the transgenic events were crushed individually into a fine powder and approximately 50 milligrams of the resulting powder was transferred to a pre-weighed centrifuge tube. The exact sample weight of the sample was recorded and 1.0 milliliter of 5% trichloroacetic acid was added to each sample tube. The samples were mixed at room temperature by vortex and then centrifuged for 15 minutes at 14,000 rpm in an Eppendorf microcentrifuge (Model 5415C, Brinkmann Instrument, Westbury, N.Y.). An aliquot of the supernatant was removed and analyzed by HPLC (Agilent 1100) using the procedure set forth in Agilent Technical Publication “Amino Acid Analysis Using the Zorbax Eclipse-AAA Columns and the Agilent 1100 HPLC” (Mar. 17, 2000), which is incorporated by reference herein. Because the R1 seeds from each event represented a population of segregating seeds, the seed with the highest tryptophan level among the 10 seeds analyzed per event was chosen as a representative of the homozygous genotype. Ten randomly selected non-transgenic seeds of Asgrow A3244 were also analyzed. The seed with the highest tryptophan level from the non-transgenic A3244 was chosen as a negative control.

### Example 10

### Effectiveness of the SAUR 3′-UTR, 2× DST, 3× DST, 4× DST, 5× DST, and 6× DST in Corn Leaf Transient Transformation

This example illustrates destabilizing sequences useful in the present invention, and their use with a gene of interest operably linked to at least one promoter element in a transgenic expression cassette of the invention. More specifically, this example demonstrates effectiveness of the SAUR 3′-UTR, 2× DST, 3× DST, 4× DST, 5× DST, and 6× DST in corn leaf transient transformation. Gene expression elements that function in dicot plants do not necessarily also function in monocot plants. The use of SAUR 3′-UTRs and DST elements to down-regulate gene expression in a dicot crop plant, soybean, is disclosed in the preceding examples of the present invention. The same elements were evaluated in a monocot, specifically a monocot crop plant (maize) to determine their effectiveness in decreasing gene expression.

In addition to SAUR 3′-UTRs and DST elements, the effectiveness of two different arrangements (overlapping AUUUA and a novel overlapping AUUUAA motifs) of overlapping repeats of an AU-rich motif was compared in a transiently transformed monocot system. In this non-limiting example, 11 copies of the AU-rich motifs were used; alternatively, either fewer (preferably at least 3) or more (greater than 11) copies could be used. Four constructs were made, two of which contained spacer sequences (random sequences of the same length) serving as controls. Synthetic complementary oligonucleotides (Invitrogen, Carlsbad, Calif.) were used to generate the spacer control sequences and the sequences of the 11 copies of AU-rich motif in two different arrangements. Oligonucleotide primers for the overlapping AUUUA arrangement were CTAGCATTTATTTATTTATTTATTTATTTATTTATTTATTTATTTATTTAG (SEQ ID NO:19) and GATCCTAAATAAATAAATAAATAAATAAATAAATAAATAAATAAATAAATG (SEQ ID NO:20). Oligonucleotide primers for the novel overlapping AUUUAA arrangement were CTAGCATTTAATTTAATTTAATTTAATTTAATTTAATTTAATTTAATTTAATTT AATTTAG (SEQ ID NO:21) and GATCCTAAATTAAATTAAATTAAATTAAATTAAATTAAATTAAATTAAATTAA ATTAAATG (SEQ ID NO:22). Oligonucleotide primers for the first spacer control were CTAGCATGAATACATCTGAATGTCTAGTATATTGATTGAAAGCTTTGTATG (SEQ ID NO:23) and GATCCATACAAAGCTTTCAATCAATATACTAGACATTCAGATGTATTCATG (SEQ ID NO:24). Oligonucleotide primers for the second spacer control were CTAGCATCTGATACTGACATGCATCATGCTAATTCAGACATGCATGAATTCAA TACGTACG (SEQ ID NO:25) and GATCCGTACGTATTGAATTCATGCATGTCTGAATTAGCATGATGCATGTCAGT ATCAGATG (SEQ ID NO:26).

These complementary oligonucleotide primer pairs were annealed together in a reaction mixture containing 1 microliter of each primer (100 micromolar), 10 microliters 10× Invitrogen buffer 10 (150 millimolar NaCl final concentration), and 88 microliters of sterile double-distilled water. The thermocycler conditions were 5 minutes at 95 degrees Celsius, followed by 70 cycles from 95 degrees Celsius (decreased 1 degree Celsius per cycle).

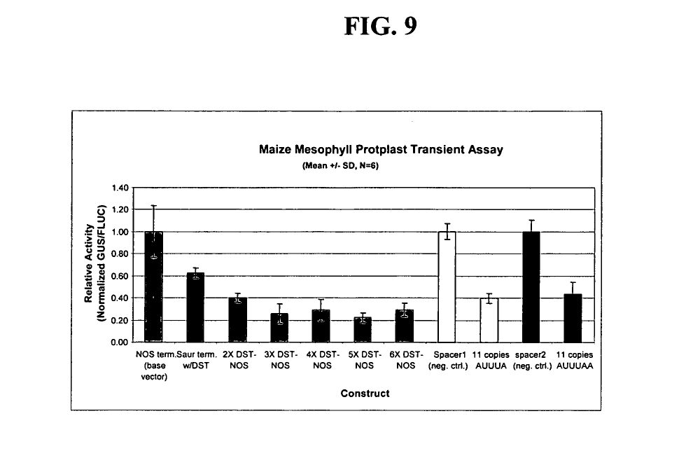

The annealed products were designed to have NheI and BamHI sites on the ends. After annealing, the products were diluted 1:50 and 1 microliter was used to ligate into the backbone of pMON64263, which had been previously cut with NheI and BamHI and gel purified. The resulting constructs were named pMON64264, pMON64265, pMON64266, and pMON64267. Corn leaf transient assays were performed with the constructs listed in Table 2 as described in Example 10. The data show that both 11-copy arrangements of the AU-rich motif resulted in lower expression (a decrease of about 50% relative to expression in the absence of an AU-rich motif repeat) (FIG. 9).

TABLE 2

| | | | Copy of DST | |
|---|---|---|---|---|
| Construct | | Coding | or AU-rich | |
| No. | Promoter | Sequence | motif | Terminator |
| pMON64256 | e35S | GUS | none | NOS |
| pMON64257 | e35S | GUS | — | SAUR |
| pMON64259 | e35S | GUS | 2X DST | NOS |
| pMON64260 | e35S | GUS | 3X DST | NOS |
| pMON64261 | e35S | GUS | 4X DST | NOS |
| pMON64262 | e35S | GUS | 5X DST | NOS |
| pMON64263 | e35S | GUS | 6X DST | NOS |
| pMON64264 | e35S | GUS | 11X AUUUA | NOS |
| pMON64265 | e35S | GUS | Spacer 1 | NOS |
| pMON64266 | e35S | GUS | 11X AUUUAA | NOS |
| pMON64267 | e35S | GUS | Spacer 2 | NOS |
| pMON19437 | e35S | Luciferase | none | NOS |
| | | (control) | | |

The promoters used in the SAUR 3′-UTR and DST constructs (pMON63691, pMON63688, pMON63697, pMON78121, pMON78120, pMON78117, pMON781116) that were analyzed in the soybean cotyledon transient assay were seed-specific promoters (FIG. 1). Other spatially-specific, temporally-specific, inducible, or constitutive promoters are also suitable. As initial constructs for evaluation in a corn leaf protoplast transient system, the seed-specific promoters of these constructs were replaced with the enhanced 35S promoter using standard molecular biological techniques. The completed constructs are shown in Table 2.

The corn leaf protoplast transient transformation experiments were performed as follows: Protoplasts were isolated from etiolated 12 day old LH200×H50 maize leaves by enzymatic digestion with 2% Cellulase RS and 0.3% Macerozyme R10 (Karlan Research, Santa Rosa, Calif.). The protoplasts were electroplorated (twice at 1 millisecond, 120 volts with 3 pulses at 5 second intervals) with 4 picomoles of plasmid DNA, which was an equal mixture of the experimental DNA and the internal control DNA (firefly luciferase, pMON 19437). Electroporations were performed in triplicate for each construct, and repeated on a different day (with at least 24 hours between the two experimental days to minimize day to day variation) for a total of six replicates. After overnight incubation, proteins were extracted by adding 0.25 volume of 5× Passive Lysing Buffer (Dual-Luciferase Reporter Assay System, catalogue number E 1960, Promega, Madison, Wis.) to the transformed protoplast cells, and 20 microliters of the protein extract were used for luciferase assay following the protocol of the Dual-Luciferase Reporter Assay System. Firefly luciferase (fLUC) activity served as the internal control. For measuring GUS activity, 20 microliters of 1× MUG (catalogue number M9130, Sigma, St. Louis, Mo.) were added to 20 microliters of the protein extract and incubated at 37 degrees Celsius for 0.5 hour. After stopping reactions by adding 180 microliters of 0.2 molar Na2CO3, fluorescence was measured with excitation at 355 nanometers and emission at 460 nanometers using a Wallac Victor2 machine (PerkinElmer, Boston, Mass.). The results (FIG. 9) demonstrated that the DST elements that resulted in gene expression at a lower level in a soybean transient system were also functional in a monocot plant system. Similar to the soy result, the degree of down-regulation was generally correlated to the copy number of DST. In this particular example, more than 3 copies of DST resulted in no further decrease in expression levels (FIG. 9), and use of a SAUR terminator containing only one copy of DST resulted in gene expression at a substantially lower level.

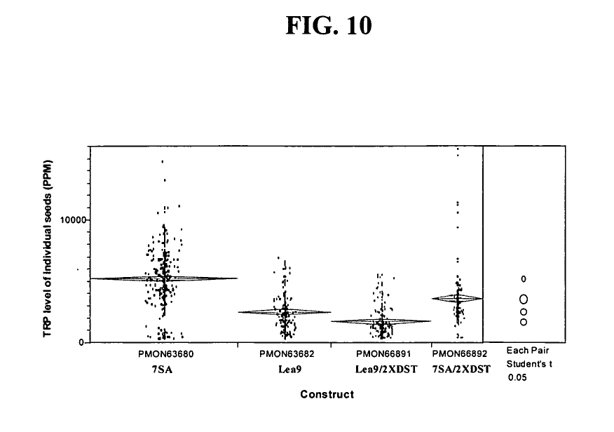

DST elements were found to be effective in lowering the tryptophan level in transgenic soybean (see Example 9). Two copies (2X) of the DST element resulted in a lowering of the tryptophan level by about 30% (FIG. 10) relative to levels observed in the absence of the DST sequence, with similar results observed for two different “parent” constructs (pMON66892 and pMON66891). Both the transiently transformed and the stably transformed plant data demonstrated that DST elements can be used to modulate gene expression in crop plants.

All of the materials and methods disclosed and claimed herein can be made and used without undue experimentation as instructed by the above disclosure. Although the materials and methods of this invention have been described in terms of preferred embodiments and illustrative examples, it will be apparent to those of skill in the art that variations may be applied to the materials and methods described herein without departing from the concept, spirit and scope of the invention. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat.: U.S. Pat. No. 4,959,317; U.S. Pat. No. 5,527,695; U.S. Pat. No. 5,538,880; U.S. Pat. No. 5,550,318; U.S. Pat. No. 5,591,616; U.S. Pat. No. 5,633,435; U.S. Pat. No. 5,750,848; U.S. Pat. No. 5,776,760; U.S. Pat. No. 5,780,708; U.S. Pat. No. 5,880,275; U.S. Pat. No. 5,837,848; U.S. Pat. No. 5,914,451; U.S. Pat. No. 5,986,175; U.S. Pat. No. 6,107,549; U.S. Pat. No. 6,118,047; U.S. Pat. No. 6,140,078; U.S. Pat. No. 6,160,208; U.S. Pat. No. 6,194,636; U.S. Pat. No. 6,232,526; U.S. Pat. No. 6,376,754; U.S. Pat. No. 6,399,861; U.S. Pat. No. 6,506,599; U.S. Pat. No. 6,583,338; U.S. Pat. No. 6,759,575; U.S. Pat. No. 5,034,322; U.S. Pat. No. 5,250,515; U.S. Pat. No. 5,763,245; U.S. Pat. No. 6,084,089; U.S. Pat. No. 6,252,138; U.S. Pat. No. 6,294,714; U.S. Pat. No. 6,426,446; U.S. Pat. No. 6,433,252; U.S. Pat. No. 6,437,217
U.S. Patent Publn. 2004/0216189
U.S. Patent Publn. 2002/0112260
U.S. Patent Publn. 2003/0106096
U.S. Patent Publn. 2004/0123347
U.S. Patent Publn. 2003/0150017 A1
Altschul et al., Nucleic Acids Res., 25:3389-3402, 1997.
Bayer and Smolke, Nature Biotechnol., 23:337-343, 2005.
Davidson and Ellington, Trends Biotechnol., 23:109-112, 2005.
Ditta et al., Proc. Natl. Acad. Sci. USA, 77:7347-7351, 1980.
Feldbrugge et al., Plant Mol. Biol., 49:215-223, 2001.
Gil et al., Plant Physiol., 104:777-784, 1994.
Green, Plant Physiol., 102:1065-1070, 1993.
Gutiérrez et al., Trends Plant Sci., 4:429-438, 1999.
Isaacs et al., Nat. Biotechnol., 22:841-847, 2004.
Maliga et al., Methods in Plant Molecular Biology, A Laboratory Course Manual, Cold Spring Harbor Laboratory Press, 1995.
Mandal and Breaker, Nature Rev. Mol. Cell Biol., 5:451-463, 2004.
Mandal and Breaker, Nature Struct. Mol. Biol., 11:29-35, 2004.
McClure et al., Plant Cell, 1:229-239, 1989.
McCown & Lloyd, Proc. Intl. Plant Propag. Soc., 30:421, 1981.
Newman et al., Plant Cell, 5:701-714, 1993,
Ohme-Takagi et al., Proc. Natl. Acad. Sci. USA, 90:11811-11815, 1993.
PCT Appln. WO 05007829
Sambrook and Russell, Molecular Cloning: A Laboratory Manual 3^{rd} Ed., Cold Spring Harbor Laboratory Press, 2001.
Sudarsan et al., RNA, 9:644-647, 2003.
Winkler et al., Nature, 419:952-956, 2002.
Yamamoto et al., Plant Cell Physiol., 33:93-97, 1992.

What is claimed is:

1. A transgenic plant comprising within its genome DNA comprising an exogenous gene encoding a polypeptide and having in its 3′ untranslated region at least a first destabilizing sequence, whereby said polypeptide is expressed at a lower level in seed of said transgenic plant relative to expression in the absence of said destabilizing sequence.

2. The transgenic plant of claim 1, wherein said transgenic plant is a crop plant selected from the group consisting of grain crop plants, oilseed crop plants, forage crop plants, and vegetable crop plants.

3. The transgenic plant of claim 1, wherein said polypeptide comprises anthranilate synthase.

4. The transgenic plant of claim 1, wherein said destabilizing sequence is selected from the group consisting of a 3′ SAUR terminator, a DST element, an ATTTA motif, an ATTTAA motif, and a combination of ATTTA and ATTTAA motifs.

5. A transgenic plant comprising within its genome DNA comprising a non-constitutive promoter operably linked to an exogenous gene encoding a polypeptide and comprising in its 3′ untranslated region at least a first destabilizing sequence, whereby said polypeptide is expressed at a lower level in said transgenic plant relative to expression in the absence of said destabilizing sequence.

6. The transgenic plant of claim 5, wherein said transgenic plant is a crop plant.

7. The transgenic plant of claim 5, wherein said non-constitutive promoter is selected from the group consisting of spatially specific promoters, temporally specific promoters, or inducible promoters.

8. The transgenic plant of claim 5, wherein said destabilizing sequence is selected from the group consisting of a 3′ SAUR terminator, a DST element, an ATTTA motif, an ATTTAA motif, and a combination of ATTTA and ATTTAA motifs.

9. A transgenic crop plant used for food or feed and comprising within its genome DNA comprising an exogenous gene encoding a polypeptide and comprising in its 3′ untranslated region a destabilizing sequence, whereby said polypeptide is expressed at a lower level in said transgenic crop plant used for food or feed relative to expression in the absence of said destabilizing sequence.

10. The transgenic crop plant used for food or feed of claim 9, wherein said transgenic crop plant used for food or feed is a monocot.

11. The transgenic crop plant used for food or feed of claim 10, wherein said monocot is maize.

12. The transgenic crop plant used for food or feed of claim 9, wherein said transgenic crop plant used for food or feed is a dicot.

13. The transgenic crop plant used for food or feed of claim 12, wherein said dicot is soybean.

14. The transgenic crop plant used for food or feed of claim 9, wherein said destabilizing sequence is selected from the group consisting of a 3′ SAUR terminator, a DST element, an ATTTA motif, an ATTTAA motif, and a combination of ATTTA and ATTTAA motifs.

15. A transgenic plant comprising within its genome DNA comprising an exogenous gene encoding a polypeptide and comprising in its 3′ untranslated region a destabilizing sequence comprising overlapping ATTTAA repeats, whereby said polypeptide is expressed at a lower level in said transgenic plant relative to expression in the absence of said destabilizing sequence.

16. A transgenic plant comprising within its genome DNA comprising a gene encoding anthranilate synthase and comprising in its 3′ untranslated region a destabilizing sequence, whereby said anthranilate synthase is expressed at a lower level in said transgenic plant relative to expression in the absence of said destabilizing sequence.

17. A method to post-transcriptionally decrease message stability of a gene of interest in a crop plant used for food or feed, comprising adding a destabilizing sequence to the 3′ untranslated region of said gene of interest in said crop plant used for food or feed, whereby message stability of said gene of interest is post-transcriptionally decreased.

18. The method of claim 17, wherein said transgenic crop plant used for food or feed is a monocot.

19. The method of claim 18, wherein said monocot is maize.

20. The method of claim 17, wherein said transgenic crop plant used for food or feed is a dicot.

21. The method of claim 20, wherein said dicot is soybean.

22. The method of claim 17, wherein said destabilizing sequence is selected from the group consisting of a 3′ SAUR terminator, a DST element, an ATTTA motif, an ATTTAA motif, and a combination of ATTTA and ATTTAA motifs.

23. The method of claim 17, wherein said gene of interest is operably linked to at least one promoter element in a transgenic expression cassette.
